(19) United States
(12) Patent Application Publication
(10) Pub. No.: US 2009/0170897 A1
(43) Pub. Date: Jul. 2, 2009

(54) Method of Treating Neuropathic Pain

(75) Inventors: Laura Corradini, Kent (GB); Mark John Field, Kent (GB); Ross Anderson Kinlock, Kent (GB); Bryn Ivor Williams-Jones, Kent (GB)

Correspondence Address: PFIZER INC., PATENT DEPARTMENT, Building 114 M/S 114, EASTERN POINT ROAD, GROTON, CT, 06340, US

(73) Assignee: Pfizer, Inc., New York, NY (US)

(21) Appl. No.: 11/568,166

(22) PCT Filed: Apr. 8, 2005
(86) PCT No.: PCT/IB2005/000992

§ 371 Date: Jun. 8, 2008

Related U.S. Application Data
(60) Provisional application No. 60/590,871, filed on Jul. 22, 2004.

(30) Foreign Application Priority Data
Apr. 20, 2004 (GB) 0408799.5

(51) Int. Cl. A61K 31/47; A61P 25/04

(52) U.S. Cl. 514/313

(57) ABSTRACT

The invention relates to the use of a CRTH2 receptor antagonist in the manufacture of a medicament for the treatment of neuropathic pain and to a method of treating neuropathic pain using an antagonist of CRTH2 receptor.

### FIELD OF THE INVENTION

The invention relates to the use of a CRTH2 receptor antagonist in the manufacture of a medicament for the treatment of neuropathic pain and to a method of treating neuropathic pain using an antagonist of CRTH2 receptor.

### BACKGROUND OF THE INVENTION

In 1999, Nagata et al identified CRTH2 (chemoattractant receptor-homologous molecule expressed on Th2 cells) also previously known as GPR44, a novel G protein-coupled receptor (GPCR) belonging to the leucocyte chemoattractant receptor family (Nagata et al., FEBS Letters (1999) 459 (2):195-9). The CRTH2 receptor is selectively expressed from a wide variety of tissues including the brain, lung and lymphoid organs in mouse (Abe et al., Gene (1999) 227 (1):71-7 ). CRTH2 receptor is selectively expressed on Th2 cells, eosinophils and basophils, but not Th1 cells, B cells and NK cells in human (Nagata et al., FEBS Letters (1999) 459 (2):195-9).

Bauer et al, (see EP1170594A2) identified Prostaglandin D₂ (PGD₂) as the endogenous ligand which is an agonist of CRTH2 receptor. PGD₂ is released from immunologically stimulated mast cells and Th2 cells. Interaction of CRTH2 with PGD₂ is known to play a critical role in the allergen-induced recruitment of Th2 cells in the target tissues of allergic inflammation. In addition, CRTH2 mediates PGD₂ dependent cell migration of blood eosinophils and basophils. Thus the CRTH2 receptor has been shown to play an active role in the molecular events providing the inflammatory and allergic response. Compounds that interfere with the PGD2-dependant activity of CRTH2 are proposed to be useful in the treatment of inflammatory and allergic disease states linked with aberrant activation of the immune system.

Torisu et al., (see WO03022814) identified indole derivatives that specifically bind to PGD2 receptors, especially the DP receptor. Since the compound binds to the CRTH2 receptor and is expected to antagonise the biological activity, it is supposed to be useful for the prevention and/or treatment of pain.

Pairaudeau et al., (see WO2004089884) disclosed substituted phenoxyacetic acids as useful pharmaceutical compounds for treating respiratory disorders, pharmaceutical compositions containing them, and processes for their preparation. Compounds are proposed to have activity as pharmaceuticals, in particular as modulators of CRTH2 receptor activity, and therefore, might be used in the treatment (therapeutic or prophylactic) of conditions/diseases in human and non-human animals which are exacerbated or caused by excessive or unregulated production of PGD2 and its metabolites; according to this document, examples of such conditions include neuropathic pain syndromes.

Surprisingly we have found that compounds that are antagonists of the CRTH2 receptor are effective in the treatment of neuropathic pain.

There are many different pain conditions, for example chronic pain, neuropathic pain, inflammatory pain, nociceptive pain, visceral pain, back pain and pain associated with disease and degeneration. The skilled person is further aware that these pain types are clinically and mechanistically distinct. Such conditions are often difficult to treat clinically due to the multiple pain symptoms. For example patients with neuropathic pain (which is a condition that can result from disease such as diabetic neuropathy or trauma to peripheral nerves or the CNS) often exhibit multiple pain symptoms including hyperlagesia (exaggerated pain to noxious stimulus), hypersensitisation, allodynia, (pain from a previously innocuous stimulus) as well as ongoing pain. Furthermore neuropathic pain is pathological as it has no protective role. It is often present well after the original cause has dissipated.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmit rapidly and are responsible for the sharp and stabbing pain sensations, whilst unmyelinated C fibres transmit at a slower rate and convey the dull or aching pain. When the injury is repaired the pain ceases. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumber facet joints, sacroiliac joints, paraspinal muscles or the posterior longitudinal ligament.

The inflammatory process is a complex series of biochemical and cellular events activated in response to tissue injury or the presence of foreign substances, which result in swelling and pain (Levine and Taiwo 1994: Textbook of Pain 45-56). Arthritic pain makes up the majority of the inflammatory pain population. Rheumatoid disease is one of the commonest chronic inflammatory conditions in developed countries and rheumatoid arthritis is a common cause of disability. The exact aetiology of RA is unknown, but current hypotheses suggest that both genetic and microbiological factors may be important (Grennan & Jayson 1994 Textbook of Pain 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder 2002 Ann Pharmacother. 36: 679-686; McCarthy et al., 1994 Textbook of Pain 387-395). Most patients with OA seek medical attention because of pain. Arthritis has a significant impact on psychosocial and physical function and is known to be the leading cause of disability in later life. Other types of inflammatory pain include but are not limited to inflammatory bowel diseases (IBD).

In contrast, the clinical characteristics of neuropathic pain are determined predominantly by the mechanisms, location, and severity of the neuropathologic process itself. Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, diabetic neuropathy, post herpetic neuralgia, back pain, cancer neuropathy, HIV neuropathy, phantom limb pain, carpal tunnel syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

Additionally, drugs conventionally used to treat nociceptive pain, such as anti-inflammatory and opiates, have limited efficacy in chronic neuropathic pain patients. Thus anti-convulsants and tricyclic antidepressant represent the main analgesics for neuropathy, despite often being poorly tolerated. In contrast to nociceptive and inflammatory pain, neuropathic pain is notoriously difficult to treat and follows a chronic course; it responds very poorly or not at all to standard analgesic therapies which are effective in the treatment of nociceptive pain such as nonsteroidal anti-inflammatory drugs and acetaminophen; and responds less predictably and less robustly to opioids than do nociceptive pain conditions. Effective treatments for nociceptive pain are not expected to extend to neuropathic pain. For instance, Gabapentin (Neurontin®) and Pregabalin (Lyrica®) reverse both static and dynamic allodynia in chronic constrictive sciatic nerve injury (CCI) and Streptozocin-induced diabetes (STZ) rat model whereas morphine reverses static but not dynamic allodynia in CCI rat model (Field M J, et al, 1999, Pain, 83: 303-311). Additionally, the efficaciousness of non-steroidal anti-inflammatory drugs (NSAIDs) and corticosteroids (dexamethason and prednisone) in chronic pain is questionable and not supported by consistent pharmacological evidence in either rodents or patients. Similarly, clinical data indicates a limited use of these medicaments in neuropathic pain disease potentially accounting for the relatively low number of rodent studies using these compounds. Schäfers (2004, Experimental Neurology, 185: 160-168) demonstrated no significant effect of non selective (Ibuprofen) and selective (Celebrex® ™) COX2 inhibitors in reversing CCI-induced pain in rats.

It is for these reasons, differences in clinical characteristics, differences in mechanism and differences in amenability to treatment, that neuropathic pain would be considered as clearly distinguished from nociceptive and inflammatory pain in the mind of the skilled person.

Accordingly, there is a critical medical need to identify pharmaceutically active compounds that interfere with key steps of the neuropathic pain processes that contribute to these pain conditions.

Additionally it is advantageous to identify target receptors involved in pain pathways which are centrally expressed in the central nervous system (CNS) and to identify pharmaceutically active compounds which exert an analgesic effect by acting centrally in the CNS and associated tissue. The CRTH2 receptor has been shown to be centrally expressed in CNS tissues including, but not necessarily restricted to the cortex, thalamus, amygdale and spinal cord as well as being expressed in a number of peripheral tissues too, (Nagata and Hirai (2003) prostaglandins, leukotrienes and Essential Fatty acids 69: 169-177). CRTH2 is also expressed in human brain and spinal cord.

Supporting tissue distribution data has been provided for the CRTH2 receptor in mouse as described in Abe et al., (1999) Gene 227: 71-77 and also in rat as described in Shichijo et al., (2003) JPET307: 518-525.

### BRIEF DESCRIPTION OF THE INVENTION

The invention is directed to the use of a CRTH2 receptor antagonist for the manufacture of a medicament for the treatment of neuropathic pain.

The present invention further provides a method of treating neuropathic pain, in a mammalian subject, which comprises administering to said subject a therapeutically effective amount of an antagonist of CRTH2 receptor.

The invention also provides a CRTH2 receptor antagonist for the treatment of neuropathic pain.

### DETAILED DESCRIPTION OF THE INVENTION

The term “CRTH2 ligand” or “CRTH2 receptor ligand” means a compound that binds to the CRTH2 receptor. Such compounds may be organic or inorganic compounds analogs or stereoisomers thereof, or other chemical or biological compounds, natural or synthesized, for example a natural prostaglandin, peptides, polypeptides, proteins, including antibodies and antibody ligand binding domains, hormones, nucleotides, nucleic acids such as DNA or RNA, and further includes a pharmaceutically acceptable salt of the compound or stereoisomer, a prodrug of the compound or stereoisomer, or a pharmaceutically acceptable salt of the prodrug. A CRTH2 receptor ligand may also be a CRTH2 receptor antagonist.

The term “CRTH2 receptor antagonist” as used herein means a compound that acts to block the activation of the CRTH2 receptor. Examples of suitable antagonists include, organic compounds such as natural prostaglandins, or analogs thereof, or other compounds, organic or inorganic molecules, peptides, proteins, including antibodies and ligand binding domains of antibodies, nucleic acids such as DNA or RNA. Suitable examples of antagonists of CRTH2 receptor may be for example organic compounds, or peptides or proteins, antibodies and fragments thereof peptidomimetic organic compounds that bind, for example, to the extra-cellular domain (ECD) of CRTH2 receptor and inhibit the activity triggered by the natural ligand. Additionally, organic compounds, peptides, antibodies or fragments thereof, to which the ECD (or a portion thereof) of CRTH2 receptor is covalently attached may also bind to and therefore “neutralize” PGD2. The term antagonist includes peptides and soluble peptides, including but not limited to members of random peptide libraries; (see, e.g., Lam et al., 1991, Nature 354:82-84; Houghten et al., 1991, Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules. Suitable antagonists may also be derived from diversity libraries, such as random or combinatorial peptide or nonpeptide, any libraries are known in the art that can be used, e.g., chemically synthesized libraries, recombinant (e.g., phage display libraries), and in vitro translation-based libraries. Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott & Smith, 1990, Science 249:386-390; Devlin et al., 1990, Science, 249:404-406; Christian, et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

Screening the libraries can be accomplished by any of a variety of commonly known methods. See, e.g., the following references, which disclose screening of peptide libraries: Parmley & Smith, 1989, Adv. Exp. Med. Biol. 251:215-218; Scott & Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar & Pabo, 1993, Science 263:671-673; and PCT Publication No. WO 94/18318.

A compound which is CRTH2 receptor antagonist may bind, and have effects, at the same site on CRTH2 receptor at which PGD₂ normally binds, although it may act at sites on CRTH2 remote to the PGD₂ binding site. Antagonists of CRTH2 receptor may act to block the CRTH2 receptor activation by any suitable means such as for example, by binding to CRTH2 receptor or to PGD2 or any other activating ligand, and thereby inhibit the binding of PGD₂ or activating ligand with CRTH2 receptor. Such antagonists may act in the place of PGD₂ at the CRTH2 receptor, or may interact with, combine with or otherwise modify PGD₂, thereby affecting how it acts at the CRTH2 receptor. Alternatively the antagonist can act to block CRTH2 receptor downstream activity for example by the modulation of CRTH2 receptor signal transduction and affecting downstream signalling events, this activity is common to inhibitors of G-proteins which can, for example, prevent the transduction of the signal as activated by PGD₂, or any other activating ligand of CRTH2 receptor. Alternatively the antagonist can act to block CRTH2 receptor activity by affecting CRTH2 receptor gene expression, such antagonists include, for example, molecules, proteins or small organic molecules or DNA or RNA, that affect transcription or interfere with splicing events so that expression of the full length or the truncated form of CRTH2 receptor can be effected. Thus such CRTH2 receptor antagonists can also include antisense RNA and sRNA products (silence interfering RNA).

Examples of suitable CRTH2 receptor antagonists for use in the invention include those compounds generally or specifically disclosed in the patent application PCT/IB 03/04505 as attached in Annex 1, in particular the compound cis-N-cyclopropyl-N-[2-methyl-1-(pyridine-3-carbonyl)-1,2,3,4-tetrahydroquinolin-4-yl]-acetamide and pharmaceutically acceptable salts and solvates thereof.

Further examples of suitable CRTH2 receptor antagonists for use in the invention include those compounds generally or specifically disclosed in the patent application WO-2004007451, 3-sulfonyl-indole derivatives and their salts, in particular the compound, 2-[5-chloro-3-(4-chlorophenylsulfonyl)-2-methyl-1H-indol-1-yl]acetic acid.

In addition, patent application WO03066047 discloses further examples of suitable CRTH2 receptor antagonists for use in the invention which are indole-3-acetic acid derivatives and their salts, in particular the compound 2-[1-(2,6-diphenoxypyrimidin-4-yl)-2,5-dimethyl-1H-indol-3-yl]acetic acid. Patent application WO03101981 discloses further suitable CRTH2 receptor antagonists, substituted indol-1-ylacetic acid derivatives, in particular the compound 3-(1,2-benzisothiazol-3-yl)-5-fluoro-2-methyl-1H-indole-1-acetic acid. WO03101961 discloses additional examples of suitable CRTH2 receptor antagonists, substituted indole compounds, in particular the compound 3-[(3-methoxyphenyl)thio]-2,5-dimethyl-1H-indole-1-acetic acid and WO03066046 discloses additional examples of suitable CRTH2 receptor antagonists, indole-3-acetic acid derivatives, in particular the compound 1-(7-chloroquinazolin-4-yl)-2-methyl-5-(1-methylethyl)-1H-indole-3-acetic acid. Further examples of suitable CRTH2 receptor antagonists include those compounds generally or specifically disclosed in the patent applications WO03097042 in particular the compound Ramatroban and WO03097598 in particular the compound (3-[1-(4-fluorobenzenesulfonyl)pyrrolidin-3-yl]indol-1-yl)acetic acid. Further examples of suitable CRTH2 receptor antagonists include antibodies or antibody subdomains to CRTH2 receptor, particularly anti CRTH2 receptor monoclonal antibody or antibody subdomains for example an antibody or subdomain specific for CRTH2 receptor, or an antibody or subdomain specific for an epitope provided in part by PGD₂.

Preferably a CRTH2 receptor antagonist according to the present invention is centrally acting. In order to be centrally acting such a compound should be able to penetrate the blood brain barrier.

A preferred CRTH2 receptor antagonist for use in the invention is a compound of general formula (I):

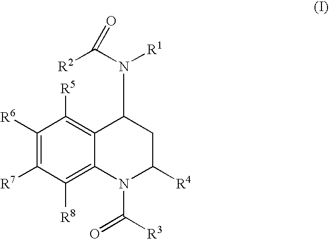

or a pharmaceutically acceptable salt or solvate thereof, wherein,

R¹ is H, (C₁-C₄)alkyl, (C₂-C₄)alkenyl, (C₂-C₄)alkynyl or (CH₂)ₘR^{x}:

R^{x} is het¹, phenyl or (C₃-C₆)cycloalkyl said het¹, phenyl and (C₃-C₆)cycloalkyl being optionally substituted by one or more Q¹ or (C₁-C₄)alkyl groups, said (C₁-C₄)alkyl being optionally substituted by one or more Q¹ groups;

Q¹ is halogen, NO₂, CN, SO₂CH₃, SO₂NR⁹R¹⁰, OR⁹, COOR⁹, C(═O)NR⁹R¹⁰, NR⁹R¹⁰, NR⁹SO₂R¹⁰, NR⁹C(═O)R¹⁰ or C(═O)R⁹ wherein R⁹ and R¹⁰ are the same or different and are selected from H and (C₁-C₄)alkyl;

m is an integer selected from 0, 1 and 2;

R² is (C₁-C₄)alkyl, wherein the alkyl group may be substituted with one or more substituents selected from halogen, OR⁹, NR⁹R¹⁰, COOR⁹, C(═O)NR⁹R¹⁰, NHSO₂R⁹ and C(═O)(C₁-C₄)alkyl wherein R⁹ and R¹⁰ are the same or different and are selected from H and (C₁-C₄)alkyl;

R³is (C₃-C₆)cycloalkyl or -A-R^{y};

A is a bond, straight or branched (C₁-C₃)alkylene, or (C₂-C₃)alkenylene;

R^{y} is (C₆-C₁₂)aryl or het², wherein the aryl and het² groups are optionally substituted by one or more substituents selected from:

(C₆-C₁₂)aryl, het¹, Q², and (C₁-C₄)alkyl, said (C₁-C₄)alkyl being optionally substituted with one or more Q² groups which are the same or different;

Q² is halogen, NO₂, CN, SO₂CH₃, SO₂NR⁹R¹⁰, OR⁹, SR⁹, OCH₂CF₃, COOR⁹, C(═O)NR⁹R¹⁰, NR⁹R¹⁰, NR⁹SO₂R¹⁰, NR⁹C(═O)R¹⁰ or C(═O)R⁹ wherein R⁹ and R¹⁰ are the same or different and are selected from H and (C₁-C₄)alkyl;

R⁴ is H or (C₁-C₄)-alkyl;

R⁵, R⁶, R⁷ and R⁸ are the same or different and are selected from H, Q³, and, (C₁-C₄)alkyl said (C₁-C₄)alkyl being optionally substituted with one or more Q³ groups which are the same or different;

Q³ is halogen, NO₂, CN, SO₂CH₃, SO₂NR⁹R¹⁰, OR⁹, SR⁹ COOR⁹, C(═O)NR⁹R¹⁰, NR⁹R¹⁰, NR⁹SO₂R¹⁰, NR⁹C(═O)R¹⁰ or C(═O)R⁹ wherein R⁹ and R¹⁰ are the same or different and are selected from H and (C₁-C₄)alkyl;

het¹ is a 5 to 10 membered aromatic heterocycle having from 1 to 4 hetero atoms selected from oxygen sulphur and nitrogen; and

het² is a 5 to 10 membered saturated, unsaturated or partially saturated heterocyclic group having from 1 to 4 hetero atoms selected from oxygen sulphur and nitrogen.

Het¹ and het² are each preferably a 5 or 6 membered aromatic heterocycle containing from 1 to 3 heteroatoms selected from oxygen, sulphur and nitrogen.

Particularly preferred definitions for het¹ and het² are isoxazolyl, oxazolyl thienyl, pyrazolyl, pyrrolyl, triazolyl, tetrazolyl, thiazolyl, isothiazolyl, thiadiazolyl, pyridinyl, pyrazinyl, benzo-oxadiazolyl or pyrazolo-pyridinyl, quinolinyl and quinoxalinyl.

(C₆-C₁₂)Aryl is understood to refer to an aromatic carbocycle containing between 6 and 12 carbon atoms. A preferred aryl group is phenyl.

The amino acid and nucleotide sequences that encode the CRTH2 receptor are known to those skilled in the art and can be found in GenBank under accession number AB008535.

Preferably a CRTH2 receptor antagonist for use in the invention is a selective CRTH2 receptor antagonist.

The term “selective” means that a ligand or antagonist binds with greater affinity to a particular receptor when compared with the binding affinity of the ligand or antagonist to another receptor. Preferably, the binding affinity of the antagonist for the first receptor is about 50% or greater than the binding affinity for the second receptor. More preferably, the binding affinity of the antagonist to the first receptor is about 75% or greater than the binding affinity to the second receptor. Most preferably, the binding affinity of the antagonist to the first receptor is about 90% or greater than the binding affinity to the second receptor. In a preferred embodiment of the invention, the antagonist exhibits a greater binding affinity for the CRTH2 receptor. Particularly preferred antagonists are those that bind with greater affinity to the CRTH receptor when compared with binding to another receptor such as a member of the chemokine receptor family for example; C3a, C5a, FMLP, LTB4, GPCR0269, GPCR0232 or GPCR0288 receptors or such as the D-type prostanoid receptor (DP), or such as the prostanoid receptor family for example prostaglandin E2 receptor subtypes EP1 to EP4, prostaglandin F receptor, thromboxane A2 receptor, most preferably DP. It is contemplated that preferred antagonists bind CRTH2 receptor with micromolar or greater affinity. More preferred antagonists bind CRTH2 receptor with nanomolar or greater affinity. Preferred CRTH2 receptor antagonists of the present invention include compounds or ligands that are selective antagonists of CRTH2 receptor. Selectivity can also be determined based on functional endpoints such as calcium mobilisation.

CRTH2 receptor ligands can be identified, for example, by screening a compound library. Methods of identifying antagonists of receptors are well known to those skilled in the art. Specific procedures that can be used to identify CRTH2 receptor ligands are presented below.

Physiological pain is an important protective mechanism designed to warn of danger from potentially injurious stimuli from the external environment. The system operates through a specific set of primary sensory neurones and is exclusively activated by noxious stimuli via peripheral transducing mechanisms (Millan 1999 Prog. Neurobio. 57: 1-164 for an integrative Review). These sensory fibres are known as nociceptors and are characterised by small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred after complex processing in the dorsal horn, either directly or via brain stem relay nuclei to the ventrobasal thalamus and then on to the cortex, where the sensation of pain is generated.

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) exaggerated pain responses to noxious stimuli (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies. Therefore pain can be divided into a number of different areas because of differing pathophysiology, these include nociceptive, inflammatory, neuropathic pain etc. It should be noted that some types of pain have multiple aetiologies and thus can be classified in more than one area, e.g. Back pain, Cancer pain have both nociceptive and neuropathic components.

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The term “therapeutically effective amount” means an amount of a compound or combination of compounds that treats a disease; ameliorates, attenuates, or eliminates one or more symptoms of a particular disease; or prevents or delays the onset of one of more symptoms of a disease.

The term “patient” means animals, such as dogs, cats, cows, horses, sheep, geese, and humans. Particularly preferred patients are mammals, including humans of both sexes.

The term “pharmaceutically acceptable” means that the substance or composition must be compatible with the other ingredients of a formulation, and not deleterious to the patient.

The terms “treating”, “treat” or “treatment” include preventative or prophylactic, and palliative treatment.

### Primary Binding Assays

CRTH2 receptor ligands and antagonists can be identified, for example by screening a compound library and by employing a variety of screening techniques. Methods of identifying ligands and antagonists of the receptor are known . Specific procedures that can be used to identify CRTH2 receptor ligands and antagonists are presented below and are recorded in the European patent application 01305857.3 (publication number EP1170594) herein incorporated by reference.

Binding assays to identify ligands of CRTH2 receptor may be performed either in the form of direct binding assays or as competition binding assays. In a direct binding assay, a test compound is tested for binding to the CRTH2 receptor. Competition binding assays, on the other hand, assess the ability of a test compound to compete with prostaglandin D₂ (PGD₂) or other suitable ligands of its family for binding to CRTH2 receptor.

In a direct binding assay, CRTH2 receptor is contacted with a test compound under conditions that allow binding of the test compound to the CRTH2 receptor. The binding may take place in solution or on a solid surface. Preferably, the test compound is previously labelled for detection. Any detectable group may be used for labelling, such as but not limited to, a luminescent, fluorescent, or radioactive isotope or group containing same, or a nonisotopic label, such as an enzyme or dye. After a period of incubation sufficient for binding to take place, the reaction is exposed to conditions and manipulations that remove excess or non-specifically bound test compound. Typically, this involves washing with an appropriate buffer. Finally, the presence a CRTH2 receptor-test compound complex is detected.

In a competition binding assay, test compounds are assayed for their ability to disrupt or enhance the binding of PGD₂ to CRTH2 receptor. Labelled PGD₂ may be mixed with CRTH2 receptor or a fragment or derivative thereof, and placed under conditions in which the interaction between them would normally occur, either with or without the addition of the test compound. The amount of labelled PGD₂ that binds CRTH2 receptor may be compared to the amount bound in the presence or absence of test compound.

In a preferred embodiment, to facilitate complex formation and detection, the binding assay is carried out with one or more components immobilized on a solid surface. In various embodiments, the solid support could be, but is not restricted to, polycarbonate, polystyrene, polypropylene, polyethylene, glass, nitrocellulose, dextran, nylon, polyacrylamide and agarose. The support configuration can include beads, membranes, microparticles, the interior surface of a reaction vessel such as a microtitre plate, test tube or other reaction vessel. The immobilization of CRTH2 receptor, or other component, can be achieved through covalent or non-covalent attachments. In one embodiment, the attachment may be indirect, i.e. through an attached antibody. In another embodiment, CRTH2 receptor and negative controls are tagged with an epitope, such as glutatione S-transferase (GST) so that the attachment to the solid surface can be mediated by a commercially available antibody such as anti-GST (Santa Cruz Biotechnology). For example, such an affinity binding assay may be performed using a CRTH2 receptor which is immobilized to a solid support. Typically, the non-immobilized component of the binding reaction, in this case either PGD₂ or the test compound, is labelled to enable detection. A variety of labelling methods are available and may be used, such as detection of luminescent, chromophoric, fluorescent, or radioactive isotopes or groups, or detection of nonisotopic labels, such as enzymes or dyes. In one preferred embodiment, the test compound is labelled with a fluorophore such as fluorescein isothiocyanate (FITC, available from Sigma Chemicals, St. Louis). The labelled test compounds, or PGD₂ plus test compounds, are then allowed to contact with the solid support, under conditions that allow specific binding to occur. After the binding reaction has taken place, unbound and non-specifically bound test compounds are separated by means of washing the surface. Attachment of the binding partner to the solid phase can be accomplished in various ways known to those skilled in the art, including but not limited to chemical cross-linking, non-specific adhesion to a plastic surface, interaction with an antibody attached to the solid phase, interaction between a ligand attached to the binding partner (such as biotin) and a ligand-binding protein (such as avidin or streptavidin) attached to the solid phase, and the like. Finally, the label remaining on the solid surface may be detected by any detection method known in the art. For example, if the test compound is labelled with a fluorophore, a fluorimeter may be used to detect complexes.

In a preferred embodiment, a binding assay can be performed as follows:

(a) Cells that express CRTH2 receptor are pelleted, and washed twice at room temperature with assay buffer (Hank's balanced saline, including Ca²⁺ and Mg²⁺, and supplemented with HEPES and sodium bicarbonate). The cells are resuspended at a concentration of 2×10⁷ cells/ml. Using 96-well U-bottom microtitre dishes, the assays are set up as follows (in 150 μl volumes):

(b) 50 μl of vehicle (as 0.3% DMSO in assay buffer, total wells); or 50 μl of 30 μM cold PGD₂ which results in a 10 μM final assay concentration thereof [the stock solution of cold PGD₂ was dissolved in DMSO at a stock concentration of 10 mM, and stored at −20° C., for use it was then diluted 3:1000 to final stock concentration of 30 μM]; 50 μl cells (2×10⁷/ml for 10⁶/well); 50 μl of 6 nM [³H]-PGD₂ is added for a final concentration of 2 nM (Amersham; 162 Ci/mmol, 0.1 Ci/ml in methanol:water:acetonitrile (3:2:1), 617 nM diluted to 10 μl per ml assay buffer for a concentration of 6 nM).

(c) The plate is allowed to incubate for 20 min at room temperature before centrifugation (2800 rpm, Sorval RT6000, 5 min, 4° C.). The supernatant is discarded to decrease non-specific binding. The plate (Packard Unifilter plate GF/C, previously soaked in 3% PEI for at least 1 hr) is harvested with cold assay buffer by washing 6 times with 150 μl buffer wash per well. The plate is dried overnight. After the addition of 50 μl scintillation fluid, the plate is counted in a scintillation counter (1 min per well). (Preferably CRTH2 receptor is added to binding assays in the form of intact cells that express CRTH2 receptor, or as isolated cell membranes that contain CRTH2 receptor. Thus, direct binding of ligand to CRTH2 receptor, or the ability of a test compound to modulate a PGD₂-CRTH2 receptor complex, may be assayed in intact cells in culture, in the presence and/or absence of the test compound). Cells that express CRTH2 receptor include 300-19 cells (transformed pre-B lymphocytes) expressing CRTH2 receptor as disclosed in M. G. Reth et al., Nature, 317(6035), pp. 353-365, 1985). CRTH2 receptor can be expressed from a plasmid which contains ampicillin and neomycin resisitance markers, and is driven by the CMV promoter. A prolac signalling peptide allows membrane expression of the gene insert, with a Flag peptide tag at the N terminal permitting convenient detection of the expressed molecule. A preferred level of expression of CRTH2 receptor is about 40,000 molecules/cell surface. A labelled PGD₂ may be mixed with cells that express CRTH2 receptor, or with crude extracts obtained from such cells, and the test compound may be added. Isolated membranes may be used to identify compounds that interact with CRTH2 receptor. For example, in a typical experiment using isolated membranes, cells may be genetically engineered to express CRTH2 receptor. Membranes can be harvested by standard techniques and used in an in vitro binding assay. Labelled ligand (e.g., ¹²⁵I-labeled PGD₂) is bound to the membranes and assayed for specific activity; and specific binding is determined by comparison with binding assays performed in the presence of excess unlabelled (cold) ligand. Alternatively, soluble CRTH2 receptor may be recombinantly expressed and utilized in non-cell based assays to identify compounds that bind to CRTH2 receptor. The recombinantly expressed CRTH2 receptor polypeptide(s) or fusion proteins containing one or more of the extra cellular domains of CRTH2 receptor can be used in the non-cell based screening assays. Alternatively, peptides corresponding to one or more of the extra cellular domains of CRTH2 receptor, or fusion proteins containing one or more of the extra cellular domains of CRTH2 receptor, can be used in non-cell based assay systems to identify compounds that bind to the cytoplasmic portion of the CRTH2 receptor; such compounds may be useful to modulate the signal transduction pathway of the CRTH2 receptor. In non-cell based assays, the recombinantly expressed CRTH2 receptor is attached to a solid substrate such as a test tube, microtitre well or a column, by means known to those in the art. The test compounds are then assayed for their ability to bind to the CRTH2 receptor.

Alternatively, the binding reaction may be carried out in solution. In this assay, the labelled component is allowed to interact with its binding partner(s) in solution. If the size differences between the labelled component and its binding partner(s) permit such a separation, the separation can be achieved by passing the products of the binding reaction through an ultrafilter whose pores allow passage of unbound labelled component but not of its binding partner(s) or of labelled component bound to its partner(s). Separation can also be achieved using any reagent capable of capturing a binding partner of the labelled component from solution, such as an antibody against the binding partner, a ligand-binding protein which can interact with a ligand previously attached to the binding partner, and so on.

The compounds of the invention are CRTH2 receptor antagonists, preferably selective CRTH2 receptor antagonists. These compounds have low IC₅₀ values, typically at least 100 nM, preferably less than 10 nM, more preferably below 1 nM.

The potency of a CRTH2 receptor antagonist (based on IC50 potency which can be defined as the concentration of antagonist that gives a halving of the value of the functional activity of a receptor in a functional assay as described below) is preferably at least 100 nM IC50 at the human receptor (recombinant and/or native), more preferably preferably less than 10 nM and further preferably less than 1 nM. For instance in a functional cell based assay, IC50 is the molar concentration of an antagonist that inhibits by 50% the maximal activation of the human CRTH2 receptor for example in response to prostaglandin D2 (or other small molecule agonists). In a binding assay, IC50 is the molar concentration of an antagonist that displaces 50% of the specific binding of ³H labelled prostaglandin D2 (or other appropriate ligand).

The selectivity of CRTH2 receptor antagonist is preferably at least 10 fold selective for CRTH2 receptor over other GPCRs especially the D type of prostanoid receptor (DP receptor), and alternatively against related members of the chemoattractant receptor subfamily for example Complement receptors C3a, C5a, FMLP (FMet-Leu-Phe receptor) FLMP-receptors I and II, Leukotriene B4 (LTB4), GPCR0269, GPCR0232, GPCR0288 receptors, preferably it should be at least 100 fold selective and further preferably at least 1000 fold selective. Selectivity in general represents the relative potency of a compound between two receptor subtypes for the appropriate ligand for the receptor of interest.

A CRTH2 receptor ligand or antagonist, can be tested for selectivity for the CRTH2 receptor in comparison with DP. In the assay, the capacity of each test compound to compete with binding of ³H-PGD₂ is measured at both the CRTH2 and DP receptors, and an IC₅₀ value (in μM) is determined. Controls can be set up using cold PGD₂ to compete with ³H-PGD₂. Any of the above mentioned binding assay procedures can be used. Selectivity of CRTH2 receptor antagonists should be at least 10 fold compared to other GPCRs especially the D type of prostanoid receptor or DP receptor (N-cyclopropyl-N-[2-methyl-1-(pyridine-3-carbonyl)-1,2,3,4-tetrahydro-quinolin-4-yl]-acetamide is >50 fold selective), preferably it should be at least 100 fold selective and even more preferably at least 1000 fold selective, alternatively the antagonist should be selective for CRTH2 receptor over any of the receptors C3a, C5a, FMLP, LTB4, GPCR0269, GPCR0232 or GPCR0288.

### Functional Assays

Functional assay methods are known for identifying a compound that modulates a CRTH2 receptor-mediated process and that are antagonists of CRTH2 receptor. The methods generally include the steps comprising: a) contacting a CRTH2 receptor-expressing cell with a test compound optionaly in the presence of PGD₂ or another CRTH2 receptor activating ligand; and b) measuring the resultant level of a CRTH2 receptor activity, or the level of expression of CRTH2 receptor in the cell, such that if said level of measured activity or expression differs from that measured in the absence of the test compound, then a compound that modulates a CRTH2 receptor-PGD2-mediated process is identified. The CRTH2 receptor activity measured can be the ability to interact with PGD₂ or the chemotactic response of the cell to PGD₂ or the intracellular Ca²⁺ concentration/mobilisation or the release of reactive oxygen species, inhibition of adenylate cyclase/cyclic AMP production or actin polymerization. Example protocols for functional assays are provided below.

Calcium mobilization can be detected and measured by flow cytometry, and by labeling with fluorescent dyes that are trapped intracellularly For example, the dye Indo-1 exhibits a change in emission spectrum upon binding calcium. The ratio of fluorescence produced by the calcium-bound dye to that produce by the unbound dye is used to estimate the intracellular calcium concentration. In an example method cells that express CRTH2 receptor are collected, and resuspended in fresh media at ˜2×10⁵/ml the day before performing the calcium flux assay. Cells are incubated at 37° C. for not longer than 20-30 minutes, and then spun down and resuspended in 50 ml fresh PTI buffer (Hank's Buffer, pH 7.2-7.4; 10 mM Hepes; 1.6 mM CaCl₂) containing Indo-1 AM, pre-warmed to 37° C., at a concentration of 10 million per ml. Cells are excited, and fluorescence is measured using a fluorimeter (Photon Technology Corporation, International). After the readout has stabilized, the time axis is reset, and PGD₂ is added at a specific time point (e.g., 20 seconds). After response, the following reagents are added to the cuvette, to release and chelate total calcium, in the following order: 20 μl of 18% Triton X-100, 20 μl of 3M Tris, pH 8.5, and 20 μl of 0.5M EGTA, pH 8.5. The experiment is repeated in the presence and the absence of a test compound. In the absence of test compound, PGD₂ results in increased ([Ca²⁺]ₗ) in cells that express CRTH2 receptor, with an EC₅₀ of 15 nM. Therefore, in the presence of an antagonist test compound, the EC₅₀ would be expected to decrease.

An actin polymerization assay may be used as a secondary screen to characterize the activity of a compound. Actin polymerization may be assayed using an actin-specific fluorescent label, nitrobenzoxadiazole (NBD)-phallacidin, which binds polymerized actin fiber. The assay may be performed as follows: cell preparations are resuspended at 5-10×10⁶ cells/ml in RPMI 1640 plus 10 mM HEPES, 100/10 Pen/Strep, and 0.5% FCS. The cell suspension is aliquoted (100 μL per well) into a 96-well U-bottom polypropylene microtiter plate. 50 ul of the appropriate stimulus (PGD₂ or test compound, or both PGD₂ and test compound) is added using an 8-channel pipette followed exactly 25 seconds later by 50 ul of a stopping solution which contains lysophosphotidylcholine (0.5 mg/ml), Hank's balanced salt solution (100 ul 10×), 16% formaldehyde (800 ul), and 6.6 uM NBD-phallacidin in MEOH (100 ul). The plate is allowed to sit at room temperature for 15 minutes. The plate is then centrifuged at 1000 rpm for 5 minutes, the supernatants flicked off and the cell pellets resuspended in 250 ul PBS plus 2% FCS and 0.2% sodium azide. Each sample is then read on a FACS Caliber instrument. Cells are gated using the forward scatter/side scatter data in the lymphocyte area. Responses are measured by the change in median FL-1 fluorescence between vehicle treated cells and stimulus treated cells. Test compounds can be assayed in the presence and absence of PGD2, and compared to a sample containing PGD₂ alone. A compound that reduces PGD₂-induced actin polymerization of CRTH2 receptor cells is identified as a candidate CRTH2 receptor antagonist.

### In Vivo Procedures

The analgesic effect of CRTH2 receptor antagonists may be determined in vivo using animal models of selected pain conditions. Several models of pain conditions are known and specific procedures that can be used to determine the analgesic effect of CRTH2 receptor antagonists are presented below.

An alternative pain model is the streptozocin induced diabetic model of neuropathic pain in rats. This procedure involves administration of streptozocin (50 mg/kg, i.p.) in a single dose to animals such as Charles River Sprague dawley rats (225-250 g) to induce diabetes. Animals are evaluated 2 weeks following administration using static and dynamic allodynia tests and if neuropathic pain is confirmed they are used to further evaluate compounds for their effect on neuropathic pain.

The chronic constrictive injury (CCI) model of pain in rats involves the tying of loose ligatures around the sciatic nerve Charles River male Sprague dawley rats (175-200 g) are placed in an anaesthetic chamber and anaesthetised with a 2% isofluorane O₂ mixture. The right hind thigh is shaved and swabbed with 1% iodine. Animals are then transferred to a homeothermic blanket for the duration of the procedure and anaesthesia maintained during surgery via a nose cone. The skin is cut along the line of the thigh bone. The common sciatic nerve is exposed at the middle of the thigh by blunt dissection through biceps femoris. Proximal to the sciatic trifurcation, about 7 mm of nerve is freed by inserting forceps under the nerve and the nerve gently lifted out of the thigh. The forceps are gently opened and closed several times to aid clearance of the fascia from the nerve. Suture is pulled under the nerve using forceps and tied in a simple knot until slight resistance is felt and then double knotted. The procedure is repeated until 4 ligatures (4-0 silk) are tied loosely around the nerve with approx 1 mm spacing. The incision is closed in layers. Fourteen days following surgery, animals are assessed for static allodynia, dynamic allodynia or weight bearing deficit.
Alternative animal models of pain conditions include the Seltzer model, partial tight ligation of the sciatic nerve (Seltzer, Z. (1995). Sem. Neurosci, 8: pp. 34-39) or Chung's model, tight ligation of one of the two spinal nerves of the sciatic nerve (Kim S H, Chung J M. Pain (1992); 50: pp. 355-63) or of the Chronic Constrictive Injury model (CCI) (Bennett G J, Xie Y-K. Pain (1988); 33: pp. 87-107). Further animal models of pain include administration of a pain inducing agent, for example Capsaicin (Dirks J, Petersen K L, Rowbotham M C, Dahl J B, Anesthesiology. 2002 July; 97(1): pp. 102-107) or Formalin (Tjolsen, A. et. al (1992), Pain 51, pp. 5-17) or Freunds Complete Adjuvant (Abdi S, Vilassova N, Decosterd I, et al, Anesth Analg 2000; 91: pp. 955-99) or Carrageenan (Itoh, M., Takasaki, I., Andoh, T., Nojima, H., Tominaga, M. & Kuraishi, Y. (2001) Neurosci. Res., 40, pp. 227-233.) or Taxol (Polomano R C. Mannes A J. Clark U S. Bennett G J, (2001) Pain. 94(3): pp. 293-304) or vinca alkaloid, vincristine (Aley K O, Reichling D B, Levine J D, Neuroscience (1996); 73: pp. 259-65) or Turpentine for visceral pain (Koster, R., Anderson, M. and De Beer, E. J., Acetic acid for analgesic screening, Fed. Proc., 18 (1959) 412./Mogil, J. S., Kest, B., Sadowski, B. and Belknap, J. K., Differential genetic mediation of sensitivity to morphine in genetic models of opiate anti-nociception: influence of nociceptive assay, J. Pharmacol. Exp. Ther., 276 (1996a) 532-544./Ness T J, Gebhart G F, Pain (1990); 41: pp. 167-234 and McMahon S B, Agents Actions (1988); 25: pp. 231-233). Further animal models of pain may involve providing to the animal a noxious physical stimulus, for example by administration of noxious heat stimulus (Malmberg, A. B., and Bannon, A. W. Models of nociception: hot-plate, tail-flick, and formalin tests in rodents. Current Protocols in Neuroscience 1999; pp 8.9.1-8.9.15) or by administration of noxious cold stimulus or noxious pressure stimulus or UV-irradiation (S. J. Boxall, A. Berthele, D. J. Laurie, B. Sommer, W. Zieglgänsberger, L. Urban and T. R. Tölle, Enhanced expression of metabotropic glutamate receptor 3 messenger RNA in the rat spinal cord during ultraviolet irradiation induced peripheral inflammation Neuroscience (1998) 82(2): pp. 591-602).

Alternative animal models of pain conditions may involve selection of an animal that naturally possesses a painful disease condition such as arthritis or HIV or Herpes or cancer or diabetes. Alternatively the animal may be arranged to experience a pain condition by modification of the animal to possess a pain inducing disease condition such as arthritis or HIV or Herpes or cancer or diabetes. Animals may be modified to possess a pain condition due to a disease in a variety of ways for example by administration of Streptozocin to induce a diabetic neuropathy (Courteix, C., Eschalier, A., Lavarenne, J., Pain, 53 (1993) pp. 81-88.) or by administration of viral proteins to cause HIV related neuropathic pain (Herzberg U. Sagen J., Journal of Neuroimmunology. (2001 May 1), 116(1): pp. 29-39) or administration of Complete Freunds Adjuvant or Mono-iodoacetate to induce arthritis and inflammatory pain (Rikard Holmdahl, Johnny C. Lorentzen, Shemin Lu, Peter Olofsson, Lena Wester, Jens Holmberg, Ulf Pettersson Immunological Reviews (2001) Volume 184, Issue 1, pp. 184) or adminstration of varicella zoster virus to cause Herpes and post herpatic neuralgia (Fleetwood-Walker S M. Quinn J P. Wallace C. Blackburn-Munro G. Kelly B G. Fiskerstrand C E. Nash A A. Dalziel R G., Journal of General Virology. 80 (Pt 9):2433-6, 1999 September) or adminstration of a carcinogen or of cancer cells to an animal to cause cancer (Shimoyama M. Tanaka K. Hasue F. Shimoyama N, Pain. 99(1-2): pp.167-74, 2002 September).

Dynamic allodynia can be assessed by lightly stroking the plantar surface of the hind paw of the animal with a cotton bud. Care is taken to perform this procedure in fully habituated rats that are not active, to avoid recording general motor activity. At least two measurements are taken at each time point, the mean of which represents the paw withdrawal latency (PWL). If no reaction is exhibited within 15 s the procedure is terminated and animals are assigned this withdrawal time. Thus, 15 s effectively represents no withdrawal. A withdrawal response is often accompanied with repeated flinching or licking of the paw. Dynamic allodynia is considered to be present if animals responded to the cotton stimulus within 8 s of commencing stroking.

Following baseline evaluation, animals can be administered compounds for analgesic assessment by one of the following routes, oral administration, subcutaneous, intra-peritoneal, intra-venous or intra-thecal. The PWL is re-evaluated at some or all of the following time points, 30 min, 1 h, 2 h, 3 h, 4 h, 5 h, 6 h, 7 h, 24 h. Animals are assigned randomly to each compound group according to their baseline values. The mean and standard error mean are calculated for each compound group at each time point. Measures of dynamic allodynia are compared to their respective controls using a one way ANOVA followed by a Dunnett's t-test comparing vehicle to compound at each time point. The minimum number of animals per group is 6.

Static allodynia can be evaluated by application of von Frey hairs (Stoelting, Wood Dale, Ill., USA) in ascending order of force (0.6, 1, 1.4, 2, 4, 6, 8, 10, 15 and 26 grams) to the plantar surface of hind paws. Animals are habituated to wire bottom test cages prior to the assessment of allodynia. Each von Frey hair is applied to the paw for a maximum of 6 seconds, or until a withdrawal response occurs. Once a withdrawal response to a von Frey hair is established, the paw is re-tested, starting with the filament below the one that produces a withdrawal, and subsequently with the remaining filaments in descending force sequence until no withdrawal occurs. The highest force of 26 g lifts the paw as well as eliciting a response, thus representing the cut off point. Each animal has both hind paws tested in this manner. The lowest amount of force required to elicit a response is recorded as paw withdrawal threshold (PWT) in grams. Static allodynia is defined as present if animals responded to a stimulus of, or less than, 4 g, which is innocuous in normal rats.

Following baseline evaluation, animals are administered compounds for analgesic assessment by one of the following routes, orally, subcutaneous, intra-peritoneal, intra-venous or intra-thecal. and the PWT re-evaluated at some or all of the following time points, 30 min, 1 h, 2 h, 3 h, 4 h, 5 h, 6 h, 7 h, 24 h. Static allodynia measurements are analysed using a Kruskall-Wallis test for non-parametric results, followed by Mann-Whitney's U test vs vehicle group. The minimum number of animals per group is 6.

Thermal hyperalgesia is assessed using the rat plantar test (Ugo Basile, Italy) following a modified method of Hargreaves et al., (1988) Pain 32:77-88. Rats are habituated to the apparatus that consists of three individual perspex boxes on an elevated glass table. A mobile radiant heat source is located under the table and focused onto the hind paw and paw withdrawal latencies (PWL) are recorded. There is an automatic cut off point of 22.5 s to prevent tissue damage. PWL are taken 2-3 times for both hind paws of each animal, the mean of which represented baselines for right and left hind paws. The apparatus is calibrated to give a PWL of approximately 10 s. PWL are reassessed 2 h following administration of carrageenan. Following administration of compounds for analgesic assessment PWL's are reassessed hourly for up to 6 hours. PWL's of compound groups are compared to their respective controls using a one way ANOVA followed by a Dunnett's t-test. The minimum number of animals per group will be 6.

Weight bearing deficit can be measured according to the method of: Bove S E, et. al. Weight bearing as a measure of disease progression and efficacy of anti-inflammatory compounds in a model of monosodium iodoacetate-induced osteoarthritis. Osteoarthritis Cartilage. 2003 Nov.; 11(11):821-30. Open field test can be carried out according to the method of Prut L and Belzung, C. The open field as a paradigm to measure the effects of compounds on anxiety-like behaviors: a review. Eur J Pharmacol. 2003; 463::3-33. The locomotor test can be carried out according to the method of Salmi P and Ahlenius S—Sedative effects of the dopamine D1receptor agonist A 68930 on rat open-field behavior. Neuroreport. 2000 Apr. 27; 11(6):1269-72.

### Combinations

A CRTH2 receptor antagonist may be usefully combined with another pharmacologically active compound, or with two or more other pharmacologically active compounds, particularly in the treatment of pain. For example, a CRTH2 receptor antagonist, particularly a compound of general formula (I), or a pharmaceutically acceptable salt or solvate thereof, as defined above, may be administered simultaneously, sequentially or separately in combination with one or more agents selected from:
(i) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
(ii) a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin or zomepirac, or a pharmaceutically acceptable salt thereof;
(iii) a barbiturate sedative, e.g. amobarbital, aprobarbital, butabarbital, butabital, mephobarbital, metharbital, methohexital, pentobarbital, phenobartital, secobarbital, talbutal, theamylal or thiopental or a pharmaceutically acceptable salt thereof;
(iv) a benzodiazepine having a sedative action, e.g. chlordiazepoxide, clorazepate, diazepam, flurazepam, lorazepam, oxazepam, temazepam or triazolam or a pharmaceutically acceptable salt thereof,
(v) an H₁ antagonist having a sedative action, e.g. diphenhydramine, pyrilamine, promethazine, chlorpheniramine or chlorcyclizine or a pharmaceutically acceptable salt thereof;
(vi) a sedative such as glutethimide, meprobamate, methaqualone or dichloralphenazone or a pharmaceutically acceptable salt thereof;
(vii) a skeletal muscle relaxant, e.g. baclofen, carisoprodol, chlorzoxazone, cyclobenzaprine, methocarbamol or orphrenadine or a pharmaceutically acceptable salt thereof,
(viii) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinone or cis-4-(phosphonomethyl)-2-piperidinecarboxylic acid or a pharmaceutically acceptable salt thereof;
(ix) an alpha-adrenergic, e.g. doxazosin, tamsulosin, clonidine or 4-amino-6,7-dimethoxy-2-(5-methanesulfonamido-1,2,3,4-tetrahydroisoquinol-2-yl)-5-(2-pyridyl) quinazoline;
(x) a tricyclic antidepressant, e.g. desipramine, imipramine, amytriptiline or nortriptiline;
(xi) an anticonvulsant, e.g. carbamazepine or valproate;
(xii) a tachykinin (NK) antagonist, particularly an NK-3, NK-2 or NK-1 antagonist, e.g. (αR,9R)-7-[3,5-bis(trifluoromethyl) benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl) phenyl]ethoxy-3-(4-fluorophenyl)-4-morpholinyl]methyl]-1,2-dihydro-3H-1,2,4-triazol-3-one (MK-869), lanepitant, dapitant or 3-[[2-methoxy-5-(trifluoromethoxy)phenyl]methylamino]-2-phenyl-piperidine (2S,3S);
(xiii) a muscarinic antagonist, e.g oxybutin, tolterodine, propiverine, tropsium chloride or darifenacin;
(xiv) a COX-2 inhibitor, e.g. celecoxib, rofecoxib or valdecoxib;
(xv) a non-selective COX inhibitor (preferably with GI protection), e.g. nitroflurbiprofen (HCT-1026);
(xvi) a coal-tar analgesic, in particular paracetamol;
(xvii) a neuroleptic such as droperidol;
(xviii) a vanilloid receptor agonist (e.g. resinferatoxin) or antagonist (e.g. capsazepine);
(xix) a beta-adrenergic such as propranolol;
(xx) a local anaesthetic, such as mexiletine;
(xxi) a corticosteriod, such as dexamethasone
(xxii) a serotonin receptor agonist or antagonist;
(xxiii) a cholinergic (nicotinic) analgesic;
(xxiv) Tramadol (trade mark);
(xxv) a PDEV inhibitor, such as sildenafil, vardenafil or taladafil;
(xxvi) an alpha-2-delta ligand such as gabapentin or pregabalin; and
(xxvii) a canabinoid.

A CRTH2 receptor antagonist is administered to a patient in a therapeutically effective amount. A CRTH2 receptor antagonist can be administered alone or as part of a pharmaceutically acceptable composition.

### Drug Substance

A CRTH2 receptor antagonist of the present invention, for example a compound of the general formula I, can be administered in the form of a pharmaceutically acceptable salt, for instance an acid addition or a base salt.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, Weinheim, Germany, 2002).

Pharmaceutically acceptable salts may be prepared by one or more of three methods:
(i) by reacting a compound with the desired acid or base;
(ii) by removing an acid- or base-labile protecting group from a suitable precursor of a compound or by ring-opening a suitable cyclic precursor, for example, a lactone or lactam, using the desired acid or base; or
(iii) by converting one salt of a compound to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288, by Haleblian (August 1975).

Hereinafter all references to a CRTH2 receptor antagonist of the present invention, for example a compound of the general formula I, include references to salts, solvates and complexes thereof and to solvates and complexes of salts thereof.

A CRTH2 receptor antagonist of the present invention, for example a compound of the general formula I, may be administered in the form of a prodrug. A prodrug is a compound which may have little or no pharmacological activity itself but which can, when administered into or onto the body, be converted into a compound having the desired activity, for example, by hydrolytic cleavage. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs can, for example, be produced by replacing appropriate functionalities present in a compound with certain moieties known to those skilled in the art as ‘pro-moieties’ as described, for example, in Design of Prodrugs by H. Bundgaard (Elsevier, 1985).

Some examples of prodrugs include
(i) where a compound contains a carboxylic acid functionality (—COOH), an ester thereof, for example, a compound wherein the hydrogen of the carboxylic acid functionality of the compound of formula (I) is replaced by (C₁-C₈)alkyl;
(ii) where a compound contains an alcohol functionality (—OH), an ether thereof, for example, a compound wherein the hydrogen of the alcohol functionality of the compound is replaced by (C₁-C₆)alkanoyloxymethyl; and
(iii) where a compound contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl.

Further examples of replacement groups in accordance with the foregoing examples and examples of other prodrug types may be found in the aforementioned references.

Moreover, certain compounds may themselves act as prodrugs of other compounds.

Also included within the scope of the invention are metabolites of a CRTH2 receptor antagonist of the present invention, for example a compound of the general formula I, that is, compounds formed in vivo upon administration of the drug. Some examples of metabolites in accordance with the invention include
(i) where a compound contains a methyl group, an hydroxymethyl derivative thereof (—CH₃->—CH₂OH):
(ii) where a compound contains an alkoxy group, an hydroxy derivative thereof (—OR->—OH);
(iii) where a compound contains a tertiary amino group, a secondary amino derivative thereof (—NR¹R²->—NHR¹ or —NHR²);
(iv) where a compound contains a secondary amino group, a primary derivative thereof (—NHR¹->—NH₂);
(v) where a compound contains a phenyl moiety, a phenol derivative thereof (-Ph->-PhOH); and
(vi) where a compound contains an amide group, a carboxylic acid derivative thereof (—CONH₂->COOH).

A CRTH2 receptor antagonist of the present invention, for example a compound of the general formula I, containing one or more asymmetric carbon atoms can exist as two or more stereoisomers. Where a compound contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds which contain an aromatic moiety. It follows that a single compound may exhibit more than one type of isomerism.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Stereoisomeric conglomerates may be separated by conventional techniques known to those skilled in the art—see, for example, Stereochemistry of Organic Compounds by E. L. Eliel and S. H. Wilen (Wiley, New York, 1994).

A CRTH2 receptor antagonist of the present invention, for example a compound of the general formula I, may exist in one or more isotopic forms wherein one or more atoms are replaced by atoms having the same atomic number, but an atomic mass or mass number different from the atomic mass or mass number which predominates in nature.

Examples of isotopes include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds can generally be prepared by conventional techniques.

Pharmaceutically acceptable solvates in accordance with the invention include those wherein the solvent of crystallization may be isotopically substituted, e.g. D₂O, d₆-acetone, d₆-DMSO.

### Drug Product

A CRTH2 receptor antagonist of the present invention, for example a compound of the general formula I, intended for pharmaceutical use may be administered as a crystalline or amorphous product. It may be obtained, for example, as a solid plug, powder, or film by methods such as precipitation, crystallization, freeze drying, spray drying, or evaporative drying. Microwave or radio frequency drying may be used for this purpose.

It may be administered alone or in combination with one or more other compounds of the invention or in combination with one or more other drugs (or as any combination thereof). Generally, it will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of a CRTH2 receptor antagonist of the present invention, for example a compound of the general formula I, and methods for its preparation will be readily apparent to those skilled in the art. Such compositions and methods for its preparation may be found, for example, in Remington's Pharmaceutical Sciences, 19th Edition (Mack Publishing Company, 1995).

### Oral Administration

A CRTH2 receptor antagonist of the present invention, for example a compound of the general formula I, may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

A CRTH2 receptor antagonist of the present invention, for example a compound of the general formula I, of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

A CRTH2 receptor antagonist of the present invention, for example a compound of the general formula I, may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, a CRTH2 receptor antagonist of the present invention, for example a compound of the general formula I, may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

### Parenteral Administration

A CRTH2 receptor antagonist of the present invention, for example a compound of the general formula I, may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of a CRTH2 receptor antagonist of the present invention, for example a compound of the general formula I, used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus a CRTH2 receptor antagonist of the present invention, for example a compound of the general formula I, may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

### Topical Administration

A CRTH2 receptor antagonist of the present invention, for example a compound of the general formula I, may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

A CRTH2 receptor antagonist of the present invention, for example a compound of the general formula I, can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of a CRTH2 receptor antagonist of the present invention, for example a compound of the general formula I, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a CRTH2 receptor antagonist of the present invention, for example a compound of the general formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. The overall daily dose may be administered in a single dose or, more usually, as divided doses throughout the day.

### Rectal/Intravaginal Administration

A CRTH2 receptor antagonist of the present invention, for example a compound of the general formula I, may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

### Ocular/Aural Administration

A CRTH2 receptor antagonist of the present invention, for example a compound of the general formula I, may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Other Technologies

A CRTH2 receptor antagonist of the present invention, for example a compound of the general formula I, may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

### Kit-Of-Parts

Inasmuch as it may desirable to administer a combination of active compounds, for example, for the purpose of treating a particular disease or condition, it is within the scope of the present invention that two or more pharmaceutical compositions, at least one of which contains a CRTH2 receptor antagonist of the present invention, for example a compound of the general formula I, may conveniently be combined in the form of a kit suitable for coadministration of the compositions.

Thus the kit of the invention comprises two or more separate pharmaceutical compositions, at least one of which contains a CRTH2 receptor antagonist of the present invention, for example a compound of the general formula I, in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

### Dosage

For administration to human patients, the total daily dose of a CRTH2 receptor antagonist of the present invention, for example a compound of the general formula I, is typically in the range 0.1 mg to 1 g depending, of course, on the mode of administration. The element of the pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsules, tablet, cachet, or lozenge itself, or it can be the appropriate number of any of these in packaged form. The quantity of active component in a unit dose preparation may be varied or adjusted from 0.1 mg to 1 g according to the particular application and the potency of the active components. In medical use the drug may be administered one to three times daily as, for example, capsules of 100 or 300 mg. In therapeutic use, the compounds utilized in the pharmaceutical method of this invention are administered at the initial dosage of about 0.01 mg to about 100 mg/kg daily. A daily dose range of about 0.01 mg to about 100 mg/kg is preferred. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

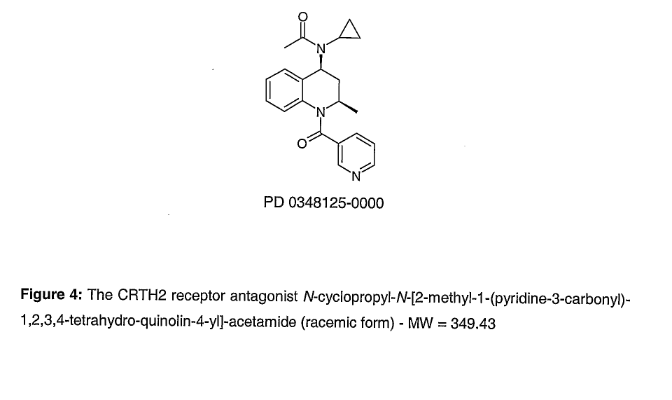

The following example illustrates the embodiments and principles of the invention and comprise the use of a potent and selective antagonist of the CRTH2 receptor N-cyclopropyl-N-[2-methyl-1-(pyridine-3-carbonyl)-1,2,3,4-tetrahydro-quinolin-4-yl]-acetamide. The structure of antagonist N-cyclopropyl-N-[2-methyl-1-(pyridine-3-carbonyl)-1,2,3,4-tetrahydro-quinolin-4-yl]-acetamide is shown in FIG. 4.

### EXAMPLES

### Animals for In Vivo Models

Male Sprague Dawley rats weighing 150-400 g obtained from Charles River (Manston, Kent, UK.) were housed in groups of 3. All animals were kept under a 12 h light/dark cycle (lights on at 07 h 00 min) with food and water ad libitum. All experiments were carried out by an observer blind to the treatments and in accordance with the Home Office Animals (Scientific Procedures) Act 1986.

### Chronic Constriction Injury (CCI) Rat Model of Neuropathic Pain

The CCI of sciatic nerve was performed as previously described by Bennett and Xie (Bennett G J, Xie Y K. A peripheral mononeuropathy in rat that produces disorders of pain sensation like those seen in man. Pain:33:87-107, 1988). Animals were anaesthetised with a 2% isofluorane/O₂ mixture. The right hind thigh was shaved and swabbed with 1% iodine. Animals were then transferred to a homeothermic blanket for the duration of the procedure and anaesthesia maintained during surgery via a nose cone. The skin was cut along the line of the thighbone. The common sciatic nerve was exposed at the middle of the thigh by blunt dissection through biceps femoris. About 7 mm of nerve was freed proximal to the sciatic trifurcation, by inserting forceps under the nerve and the nerve gently lifted out of the thigh. Suture was pulled under the nerve using forceps and tied in a simple knot until slight resistance was felt and then double knotted. The procedure was repeated until 4 ligatures (4-0 silk) were tied loosely around the nerve with approx 1 mm spacing. The incision was closed in layers.

### Streptozocin (STZ)-Induced Diabetes Neuropathy in the Rat

Diabetes was induced by a single intraperitoneal injection of streptozotocin (50 mg/kg) freshly dissolved in 0.9% sterile saline. Streptozotocin injection induces a reproducible mechanical allodynia within 3 weeks, lasting for at least 7 weeks (Chen and Pan, (Chen S R and Pan H L. Hypersensitivity of Spinothalamic Tract Neurons Associated With Diabetic Neuropathic Pain in Rats. J Neurophysiol 87: 2726-2733, 2002).

### Assessment of Static and Dynamic Allodynia in the Rat

### Static Allodynia

Animals were habituated to wire bottom test cages prior to the assessment of allodynia. Static allodynia was evaluated by application of von Frey hairs (Stoelting, Wood Dale, Ill., USA.) in ascending order of force (0.6, 1, 1.4, 2, 4, 6, 8, 10, 15 and 26 grams) to the plantar surface of hind paws. Each von Frey hair was applied to the paw for a maximum of 6 sec, or until a withdrawal response occurred. Once a withdrawal response to a von Frey hair was established, the paw was re-tested, starting with the filament below the one that produced a withdrawal, and subsequently with the remaining filaments in descending force sequence until no withdrawal occurred. The highest force of 26 g lifted the paw as well as eliciting a response, thus represented the cut off point. Each animal had both hind paws tested in this manner. The lowest amount of force required to elicit a response was recorded as paw withdrawal threshold (PWT) in grams. Static allodynia was defined as present if animals responded to a stimulus of, or less than, 4 g, which is innocuous in naive rats (Field M J, Bramwell S, Hughes J, Singh L. Detection of static and dynamic components of mechanical allodynia in rat models of neuropathic pain: are they signalled by distinct primary sensory neurones? Pain, 1999; 83:303-11).

### Dynamic Allodynia

Dynamic allodynia was assessed by lightly stroking the plantar surface of the hind paw with a cotton bud. To avoid recording general motor activity, care was taken to perform this procedure in fully habituated rats that were not active. At least two measurements were taken at each time point, the mean of which represented the paw withdrawal latency (PWL). If no reaction was exhibited within 15 sec the procedure was terminated and animals were assigned this withdrawal time. A pain withdrawal response was often accompanied with repeated flinching or licking of the paw. Dynamic allodynia was considered to be present if animals responded to the cotton stimulus within 8 sec of commencing stroking (Field et al, 1999).

### Carrageenan-Induced Thermal Hyperalgesia (CITH) in the Rat

Thermal hyperalgesia was assessed using the rat plantar test (Ugo Basile, Comerio, Italy), according to a method modified by Hargreaves et al. (1988). Briefly, rats were habituated to the apparatus that consisted of three individual Perspex boxes on a glass table. A mobile radiant heat source was located under the table and focused onto the desired paw. Paw withdrawal latencies (PWLs) were recorded three times for both hind paws of each animal, the mean of which represented baseline for left and right hind paws. The apparatus was calibrated to give a PWL of approximately 10 s in naïve rats. To prevent tissue damage of the plantar zone, a 22.5 sec cut-off was observed. Lambda carrageenan was injected intraplantarly (100 μl, 20 mg/ml) the right hind paw and baseline recordings of PWT were taken 2 hr post administration.

### Data Analysis

All the experiments were conducted blind. Static allodynia was expressed as median [LQ; UQ] and analysed by Mann Whitney U test. Dynamic allodynia and thermal hyperalgesia were expressed as arithmetic mean±SEM and analysed by ANOVA.

### Effect of N-cyclopropyl-N-[2-methyl-1-(pyridine-3-carbonyl)-1,2,3,4-tetrahydro-quinolin-4-yl]-acetamide on CCI-Induced Static and Dynamic Allodynia

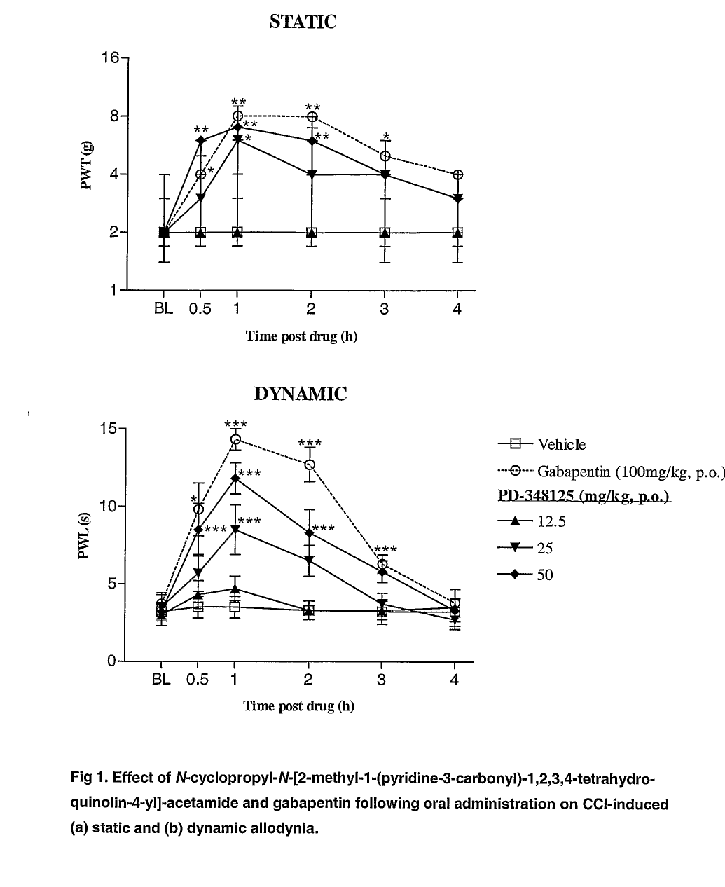

Naïve rats exhibit paw withdrawal thresholds of approximately 10 g to von Frey application and find application of a cotton bud stimulus completely innocuous. Following nerve injury rats display increased sensitivity to both of these stimuli indicating the development of static and dynamic allodynia. From 14 days post surgery animals exhibited typical static and dynamic allodynic responses and the baseline recorded before the test were <4 g and <4 sec, respectively in all animals. These allodynic responses remained consistent throughout the experiments in the vehicle-treated group. Following oral (PO) administration, N-cyclopropyl-N-[2-methyl-1-(pyridine-3-carbonyl)-1,2,3,4-tetrahydro-quinolin-4-yl]-acetamide (12.5, 25 and 50 mg/kg) reversed the maintenance of CCI-induced static and dynamic allodynia in a dose dependent manner (FIG. 1A and FIG. 1B). The MED was 25 mg/kg and produced a peak effect at 1 hr post administration in both static and dynamic allodynia. The highest dose showed an anti-allodynic effect in both behavioral tests from 30 min post dose (p<0.01 vs vehicle-treated group). It reversed static allodynia with a curve profile comparable to gabapentin (100 mg/kg, PO) while its effect in dynamic allodynia is less potent but significantly different from vehicle treated CCI rats (11.8±1.0 vs 3.5±0.7 at 2 hrs post administration).

### Effect of N-cyclopropyl-N-[2-methyl-1-(pyridine-3-carbonyl)-1,2,3,4-tetrahydro-quinolin-4-yl]-acetamide on STZ-Induced Static and Dynamic Allodynia

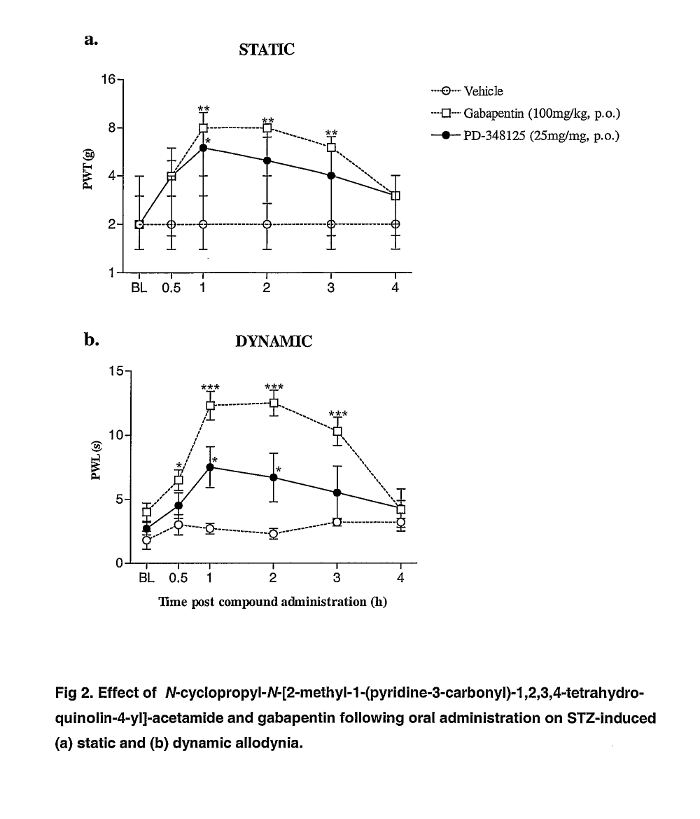

Naïve rats exhibit paw withdrawal thresholds of approximately 10 g to von Frey application and find application of a cotton bud stimulus completely innocuous. Following streptozocin injection rats display increased sensitivity to both of these stimuli indicating the development of static and dynamic allodynia. From day 14 post STZ injection rats were selected based on their pain-like threshold (PWT and PWL) and used for pharmacological studies. Baseline readings in all animals were <4 g and <5 sec for static and dynamic allodynia, respectively (FIG. 2A and FIG. 2B). These allodynic responses remained consistent throughout the experiments in the vehicle treated group. Following N-cyclopropyl-N-[2-methyl-1-(pyridine-3-carbonyl)-1,2,3,4-tetrahydro-quinolin-4-yl]-acetamide (25mg/kg, PO) administration the maintenance of both STZ-induced static and dynamic allodynia was reversed. The peak effect was seen at 1 hr post compound dose and was biologically relevant up to 2 hr. Gabapentin (100 mg/kg, PO), which was included in the experiment as a positive control produced a complete reversal of both static and dynamic allodynia end points.

### Effect of N -cyclopropyl-N-[2-methyl-1-(pyridine-3-carbonyl)-1,2,3,4-tetrahydro-quinolin-4-yl]-acetamide on CITH in the Rat

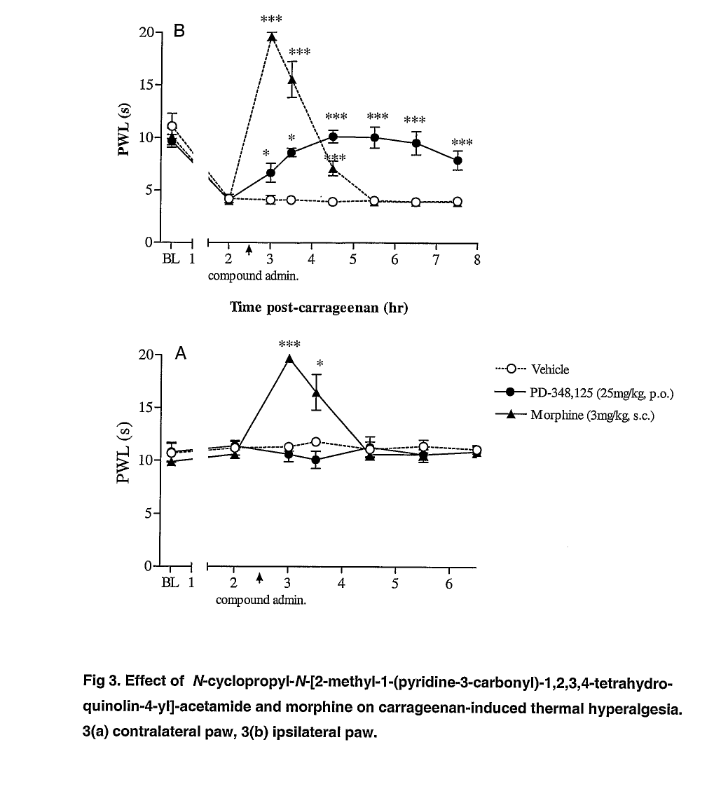

Naïve rats exhibit paw withdrawal latencies (PWL) of approximately 10 sec to thermal stimulation. Two hours following unilateral intraplantar injection of carrageenan rats increased sensitivity to thermal stimuli indicating the development of thermal hyperalgesia in the ipsilateral paw (mean of baseline 11.0±0.5 and 4.1±0.3 sec for contra and ipsilateral paw respectively). These PWT remained consistent throughout the time course in the vehicle-treated group (FIG. 3B). N-cyclopropyl-N-[2-methyl-1-(pyridine-3-carbonyl)-1,2,3,4-tetrahydro-quinolin-4-yl]-acetamide (25 mg/kg, PO) completely reversed the maintenance of thermal hyperalgesia with a peak effect at 2 h post administration (10.1±0.6 vs 3.9±0.2 for vehicle treated group). This anti-hyperalgesic effect remained consistent for 5 h post compound administration and no effect was observed in the contralateral paw (FIG. 3A). Morphine (3 mg/kg, SC), which was included in the experiment as a positive control, produced the expected analgesic effect. It increased the PWLs in both hindpaws over the baseline value of naïve rats at 30 min post dose.

### Discussion

The present study demonstrates that a selective CRTH2 receptor (CRTH2R) antagonist, can reverse static and dynamic allodynia in the chronic constriction injury and STZ-induced diabetic animal models of neuropathy. Moreover the antagonist produced a long lasting anti-hyperalgesic effect in the CITH rat model. N-cyclopropyl-N-[2-methyl-1-(pyridine-3-carbonyl)-1,2,3,4-tetrahydro-quinolin-4-yl]-acetamide was dosed in the rat blood and CSF at various time points after oral administration. At 4 hr post injection, more than 5×IC50 (rIC50=45 nM) was measured in the cerebro spinal fluid (CSF) of naïve animals orally treated with 25 mg/kg of compound. Therefore, the anti-hyperalgesic profile observed in CITH rat model does represent the picture of a centrally active compound, the compound appears to cross the blood brain barrier to act centrally at the receptor.

CRTH2R antagonist, N-cyclopropyl-N-[2-methyl-1-(pyridine-3-carbonyl)-1,2,3,4-tetrahydro-quinolin-4-yl]-acetamide shows efficacy in animal models of neuropathic pain.

In conclusion, the CRTH2 receptor antagonist, N-cyclopropyl-N-[2-methyl-1-(pyridine-3-carbonyl)-1,2,3,4-tetrahydro-quinolin-4-yl]-acetamide, reverses static and dynamic allodynia in two rodent models of neuropathy, specifically the rat Chronic constriction injury (CCI) of the sciatic nerve and rat streptozotocin (STZ)-induced diabetes (Field M J, et al, 1999, Pain, 83: 303-311).

In the same animal models, the effect of N-cyclopropyl-N-[2-methyl-1-(pyridine-3-carbonyl)-1,2,3,4-tetrahydro-quinolin-4-yl]-acetamide is comparable to Gabapentin, the current market-leading drug for the treatment of neuropathic pain.

This experimental evidence suggests that CRTH2 receptor antagonists are efficacious in the treatment of human neuropathic pain.

1. (canceled)

2. (canceled)

3. (canceled)

4. (canceled)

5. (canceled)

6. (canceled)

7. A method of treating neuropathic pain, in a mammalian subject, which comprises administering to said subject a therapeutically effective amount of an antagonist of CRTH2 receptor.

8. A method of treatment as claimed in claim 7 wherein the CRTH2 receptor antagonist is a compound of general formula (I): 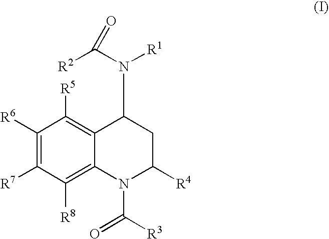
or a pharmaceutically acceptable salt or solvate thereof, wherein,
R¹ is H, (C₁-C₄)alkyl, (C₂-C₄)alkenyl, (C₂-C₄)alkynyl or (CH₂)ₘR^{x}:
R^{x} is het¹, phenyl or (C₃-C₆)cycloalkyl said het¹, phenyl and (C₃-C₆)cycloalkyl being optionally substituted by one or more Q¹ or (C₁-C₄)alkyl groups, said (C₁-C₄)alkyl being optionally substituted by one or more Q¹ groups;
Q¹ is halogen, NO₂, CN, SO₂CH₃, SO₂NR⁹R¹⁰, OR⁹, COOR⁹, C(═O)NR⁹R¹⁰, NR⁹R¹⁰, NR⁹SO₂R¹⁰, NR⁹C(═O)R¹⁰ or C(═O)R⁹ wherein R⁹ and R¹⁰ are the same or different and are selected from H and (C₁-C₄)alkyl;
m is an integer selected from 0, 1 and 2;
R² is (C₁-C₄)alkyl, wherein the alkyl group may be substituted with one or more substituents selected from halogen, OR⁹, NR⁹R¹⁰, COOR⁹, C(═O)NR⁹R¹⁰, NHSO₂R⁹ and C(═O)(C₁-C₄)alkyl wherein R⁹ and R¹⁰ are the same or different and are selected from H and (C₁-C₄)alkyl;
R³ is (C₃-C₆)cycloalkyl or -A-R^{y};
A is a bond, straight or branched (C₁-C₃)alkylene, or (C₂-C₃)alkenylene;
R^{y} is (C₆-C₁₂)aryl or het², wherein the aryl and het² groups are optionally substituted by one or more substituents selected from,
(C₆-C₁₂)aryl, het¹, Q², and (C₁-C₄)alkyl, said (C₁-C₄)alkyl being optionally substituted with one or more Q²groups which are the same or different;
Q² is halogen, NO₂, CN, SO₂CH₃, SO₂NR⁹R¹⁰, OR⁹, SR⁹, OCH₂CF₃, COOR⁹, C(═O)NR⁹R¹⁰, NR⁹R¹⁰, NR⁹SO₂R¹⁰, NR⁹C(═O)R¹⁰ or C(═O)R⁹ wherein R⁹ and R¹⁰ are the same or different and are selected from H and (C₁-C₄)alkyl;
R⁴ is H or (C₁-C₄)-alkyl;
R⁵, R⁶, R⁷ and R⁸ are the same or different and are selected from H, Q³, and, (C₁-C₄)alkyl said (C₁-C₄)alkyl being optionally substituted with one or more Q³ groups which are the same or different;
Q³ is halogen, NO₂, CN, SO₂CH₃, SO₂NR⁹R¹⁰, OR⁹, SR⁹ COOR⁹, C(═O)NR⁹R¹⁰, NR⁹R¹⁰, NR⁹SO₂R¹⁰, NR⁹C(═O)R¹⁰ or C(═O)R⁹ wherein R⁹ and R¹⁰ are the same or different and are selected from H and (C₁-C₄)alkyl;
het¹ is a 5 to 10 membered aromatic heterocycle having from 1 to 4 hetero atoms selected from oxygen sulphur and nitrogen; and
het² is a 5 to 10 membered saturated, unsaturated or partially saturated heterocyclic group having from 1 to 4 hetero atoms selected from oxygen sulphur and nitrogen.

9. A method of treatment as claimed in claim 8, wherein the CRTH2 receptor antagonist is cis-N-cyclopropyl-N-[2-methyl-1-(pyridine-3-carbonyl)-1,2,3,4-tetrahydro-quinolin-4-yl]-acetamide, or a pharmaceutically acceptable salt or solvate thereof.

10. A method of treatment as claimed in claim 7, wherein the CRTH2 receptor antagonist is an antibody, an antibody ligand binding domain or a polynucleotide.

11. A method of treatment as claimed in claim 7 wherein the CRTH2 receptor antagonist is used separately, sequentially or simultaneously in combination with a second pharmacologically active compound.

12. A method of treatment as claimed in claim 11 wherein the second pharmacologically active compound is selected from;
(xix) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
(xx) a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin or zomepirac, or a pharmaceutically acceptable salt thereof;
(xxi) a barbiturate sedative, e.g. amobarbital, aprobarbital, butabarbital, butabital, mephobarbital, metharbital, methohexital, pentobarbital, phenobartital, secobarbital, talbutal, theamylal or thiopental or a pharmaceutically acceptable salt thereof;
(xxii) a benzodiazepine having a sedative action, e.g. chlordiazepoxide, clorazepate, diazepam, flurazepam, lorazepam, oxazepam, temazepam or triazolam or a pharmaceutically acceptable salt thereof,
(xxiii) an H₁ antagonist having a sedative action, e.g. diphenhydramine, pyrilamine, promethazine, chlorpheniramine or chlorcyclizine or a pharmaceutically acceptable salt thereof;
(xxiv) a sedative such as glutethimide, meprobamate, methaqualone or dichloralphenazone or a pharmaceutically acceptable salt thereof;
(xxv) a skeletal muscle relaxant, e.g. baclofen, carisoprodol, chlorzoxazone, cyclobenzaprine, methocarbamol or orphrenadine or a pharmaceutically acceptable salt thereof,
(xxvi) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinone or cis-4-(phosphonomethyl)-2-piperidinecarboxylic acid or a pharmaceutically acceptable salt thereof;
(xxvii) an alpha-adrenergic, e.g. doxazosin, tamsulosin, clonidine or 4-amino-6,7-dimethoxy-2-(5-methanesulfonamido-1,2,3,4-tetrahydroisoquinol-2-yl)-5-(2-pyridyl) quinazoline;
(xxviii) a tricyclic antidepressant, e.g. desipramine, imipramine, amytriptiline or nortriptiline;
(xxix) an anticonvulsant, e.g. carbamazepine or valproate;
(xxx) a tachykinin (NK) antagonist, particularly an NK-3, NK-2 or NK-1 antagonist, e.g. (□R,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant or 3-[[2-methoxy-5-(trifluoromethoxy)phenyl]methylamino]-2-phenyl-piperidine (2S,3S);
(xxxi) a muscarinic antagonist, e.g oxybutin, tolterodine, propiverine, tropsium chloride or darifenacin;
(xxxii) a COX-2 inhibitor, e.g. celecoxib, rofecoxib or valdecoxib;
(xxxiii) a non-selective COX inhibitor (preferably with GI protection), e.g. nitroflurbiprofen (HCT-1026);
(xxxiv) a coal-tar analgesic, in particular paracetamol;
(xxxv) a neuroleptic such as droperidol;
(xxxvi) a vanilloid receptor agonist (e.g. resinferatoxin) or antagonist (e.g. capsazepine);
(xix) a beta-adrenergic such as propranolol;
(xx) a local anaesthetic, such as mexiletine;
(xxi) a corticosteriod, such as dexamethasone
(xxii) a serotonin receptor agonist or antagonist;
(xxiii) a cholinergic (nicotinic) analgesic;
(xxiv) Tramadol (trade mark);
(xxv) a PDEV inhibitor, such as sildenafil, vardenafil or taladafil;
(xxvi) an alpha-2-delta ligand such as gabapentin or pregabalin; and
(xxvii) a canabinoid.
